(19) United States
(12) Patent Application Publication
(10) Pub. No.: US 2025/0160799 A1
(43) Pub. Date: May 22, 2025

(54) CARDIAC FUNCTION ASSESSMENT AND CLASSIFICATION

(71) Applicant: Worcester Polytechnic Institute, Worcester, MA (US)

(72) Inventors: Abdulsalam Almadani, Worcester, MA (US); Emmanuel Agu, Worcester, MA (US); Abhishek Shivdeo, Worcester, MA (US); Atifa Sarwar, Worcester, MA (US); Jacques Kpodonu, Worcester, MA (US); Monica Ahluwalia, Worcester, MA (US)

(21) Appl. No.: 18/955,638

(22) Filed: Nov. 21, 2024

Related U.S. Application Data
(60) Provisional application No. 63/601,605, filed on Nov. 21, 2023.

(51) Int. Cl. A61B 8/08; A61B 8/14

(52) U.S. Cl. CPC A61B 8/5223; A61B 8/0883; A61B 8/14; A61B 2576/023

(57) ABSTRACT

A cardiovascular analysis application receives patient echocardiograms as video segments and performs an analysis for cardiovascular health based on factors related to an ejection fraction and hypertrophic cardiomyopathy based on the images in the video segment and spatiotemporal features extracted from the images through several heartbeat cycles on the video segments. The models are trained on a corpus of previous echocardiograms including labels indicative of the ejection fraction and physiological markers associated with HCM, such as the cardiac wall thickness and clarity. Based on a correspondence with the model, a result is rendered indicative of whether the patient video segment has an insufficient ejection fraction and whether a presence of HCM is exhibited.

### RELATED APPLICATIONS

This patent application claims the benefit under 35 U.S.C. § 119 (e) of U.S. Provisional Patent App. No. 63/601,605, filed Nov. 21, 2023, entitled “CARDIAC FUNCTION ASSESSMENT AND CLASSIFICATION,” incorporated herein by reference in entirety.

### BACKGROUND

Cardiovascular diseases (CVDs) are a major contributor to mortality, causing 18.6 million deaths globally in 2019. The number of CVD cases increased from 271 million in 1990 to 523 million in 2019 with CVDs. Medical imaging techniques, especially echocardiography (ECD), which is an ultrasound of the heart that enables visualization of the heart's activity, are indispensable for detecting, diagnosing, risk stratification, hemodynamic assessment, and interventional management of various cardiac conditions.

### SUMMARY

A cardiovascular analysis application receives patient echocardiograms as video segments and performs an analysis for cardiovascular health based on factors related to an ejection fraction and hypertrophic cardiomyopathy (HCM) based on the images in the video segment and spatiotemporal features extracted from the images through several heartbeat cycles on the video segments. Classification models are trained on a corpus of previous echocardiograms including labels indicative of the ejection fraction and physiological markers associated with HCM, such as the cardiac wall thickness and clarity. Based on a correspondence with the models, a result is rendered indicative of whether the patient video segment has an insufficient ejection fraction and whether a presence of HCM is exhibited.

Configurations herein are based, in part, on the observation that medical imaging is often employed for analysis and diagnosis of internal anatomical structures, particularly soft tissue organs that may not appear well on older mediums such as an X-ray. Unfortunately, imaging approaches, particularly 2-dimensional scans, can be burdensome to properly analyze and do not show changes over time such as movement of a beating heart. Accordingly, configurations herein substantially overcome the shortcomings of conventional approaches to heart evaluation by providing a model of echocardiograms trained from video segments of heartbeat cycles, and classifying a patient echocardiogram using the trained model for features associated with an insufficient ejection fraction and features indicative of HCM.

In further detail, configurations herein provide a method for analysis of echocardiograms for determining health of a human heart by receiving a video segment based on an echocardiogram, and comparing the video segment to a model of echocardiograms, where the model is trained using labels for a sufficiency of an ejection fraction and a likelihood of hypertrophic cardiomyopathy (HCM). The system then renders an indication of a presence of HCM and an indication of an insufficient ejection fraction based on the classification.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a visualization of the Apical 4-Chamber (A4C) view of an echocardiogram as employed herein;

FIGS. 2A and 2B show electrocardiogram differences between healthy and unhealthy hearts;

FIG. 3 is a system context diagram including an echocardiogram gathering device and an analysis computer;

FIGS. 4A-4C show scans exhibiting features employed for classification, and for outlier/border scenarios where artifacts and anomalies can contribute to false positives/negatives

FIG. 5 shows a block diagram of the EF classification pipeline;

FIGS. 6A-6D show examples of echocardiogram views exemplary of the HCM training set; and

FIG. 7 shows a block diagram of the HCM classification pipeline.

### DETAILED DESCRIPTION

The description below presents an example of training and classification using a model of heart echocardiograms for assessing a likelihood of deficient heart conditions such as a compromised ejection fraction and HCM.

Cardiovascular diseases are a major and growing contributor to global mortality. The Ejection Fraction (EF) of a heart, defined as the ratio of the amount of blood pumped out of the heart-ventricle to the volume of blood entering the ventricle at the start of each heartbeat, is an important parameter for assessing cardiac function. The echocardiogram, an ultrasound that generates videos of the beating heart, is widely used to assess cardiac function and diagnose CVDs. Interpretation of echocardiograms is currently done by experts, who are not always available in many low resource settings and can be inconsistent and make errors. Video Action Recognition (VAR) Neural Networks have recently been proposed to recognize the actions being performed by humans in videos. Configurations of the disclosed approach implement VAR neural networks for the binary classification of echocardiograms into healthy and unhealthy EF classes. A Gate Shift Network (GSN) with BNInception as its provides a superior VAR architecture, achieving an accuracy of 90.17% and a specificity of 77.01% with an inference time as low as 25.11 seconds.

Hypertrophic Cardiomyopathy (HCM) is the most common genetic disease in which the heart's Left Ventricular (LV) wall becomes thicker and stiffer, making it difficult to pump blood. HCM affects 1:200 to 1:500 people and can result in Sudden Cardiac Death (SCD), heart failure, and abnormal heart rhythms leading to stroke. Early diagnosis and treatment of HCM can improve outcomes. An echocardiogram is routinely performed on patients and is currently the gold standard for HCM diagnosis. However, expert analyses of echocardiograms can be inconsistent, resulting in missed diagnoses. Deep Video Action Recognition (VAR) models are an ideal medium for the task of recognizing human actions, such as running and walking, in a video format suitable for spatiotemporal analysis.

Vector autoregression (VAR) is a statistical model used to capture the relationship between multiple quantities as they change over time. VAR is a type of stochastic process model. VAR models generalize the single-variable (univariate) autoregressive model by allowing for multivariate time series.

A heart's Ejection Fraction (EF) is an important parameter for assessing its function. EF is defined as the ratio of the amount of blood pumped out of the heart's left ventricle (End Diastolic Volume (EDV)) to the volume of blood entering the ventricle (End Systolic Volume (ESV)) at the start of each heartbeat, expressed in the equation below. Healthy hearts generally have EF values of above 50%.

$$Ejection Fraction ( EF ) ( \% ) = \frac{EDV - ESV}{EDV} \times 100$$

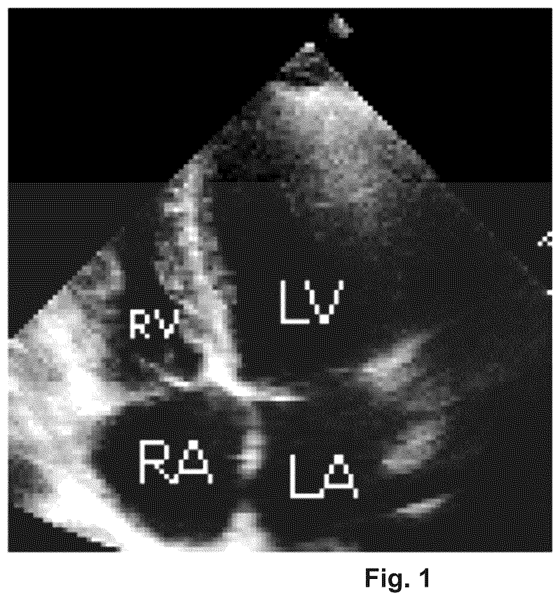

FIG. 1 is a visualization of the Apical 4-Chamber (A4C) view of an echocardiogram, showing the Left Ventricle (LV), Right Ventricle (RV), Left Atrium (LA), and Right Atrium (RA) in the echocardiogram.

EF can be assessed from echocardiograms, which facilitates early diagnoses and treatment of CVDs, and prevent heart failure. However, expert interpretation and estimation of the EF from an echocardiogram can be time-consuming, inconsistent across evaluators, and prone to errors. Moreover, this task is typically performed by a trained cardiologist with several years of training and expertise, who may not be available in low resource settings. Machine learning (ML) and deep learning (DL) approaches can automate echocardiography analyses, improving the consistency of interpretation, detection of cardiac pathologies and assessment of cardiac function including EF. Some prior work utilized machine learning classification or regression of echocardiography videos or static images to predict various ailments such as HCM, estimate important cardiac attributes such as EF value and determine echocardiogram views such as A2C (Apical 2-Chamber), A4C and others.

Video Action Recognition (VAR) Neural Networks have recently been proposed to recognize the actions (e.g. walking, riding a bike) being performed by humans in videos. VAR models have been explored for diverse tasks including surveillance and robotics. The disclosed approach utilizes a comprehensive VAR deep learning model for binary classification (healthy vs. unhealthy EF) of echocardiogram videos. Specifically, configurations herein employ a Gate Shift Network (GSN) for binary classification of EF in echocardiograms. The GSN architecture uses the Gate Shift Module (GSM) as its building block. GSM is a lightweight module that decomposes 3D convolutions spatially and temporally using learnable spatial gating blocks, allowing it to learn complex features with a minimal number of parameters.

Configurations disclosed herein builds on an approach of analyzing echo videos directly to learn both spatial and temporal features instead of analyzing still images of echocardiogram views using 2D or 3D CNN (convolutional neural network) architectures that was utilized in much of prior work. Still images do not capture important temporal beat-to-beat information such as myocardial motion, heart rate and heart rhythm. Analyzing transthoracic echocardiogram (TTE) videos directly has been found by prior work to outperform analyzing still image views. Conventional approaches have analyzed video directly to predict CVDs including post-operative right ventricular failure (AUC of 0.729) (AUC stands for “Area Under the ROC Curve” and is a metric used in cardiology to measure the performance of tests that distinguish between diseased and nondiseased individuals), anemia (AUC of 0.8), elevated B-type natriuretic peptide (BNP) (AUC of 0.82), elevated troponin (AUC of 0.75) and elevated blood urea nitrogen (BUN) (AUC of 0.69). Another benefit of directly analyzing videos is that as it obviates the need to first detect the cardiac view before using view-dependent ML models to detect various cardiac conditions, a labeling-intensive task. For instance, a 3D cardiac structure captured by a single video corresponds to still images from 70 different viewpoints in a 2D TTE, whose labels are not always readily available.

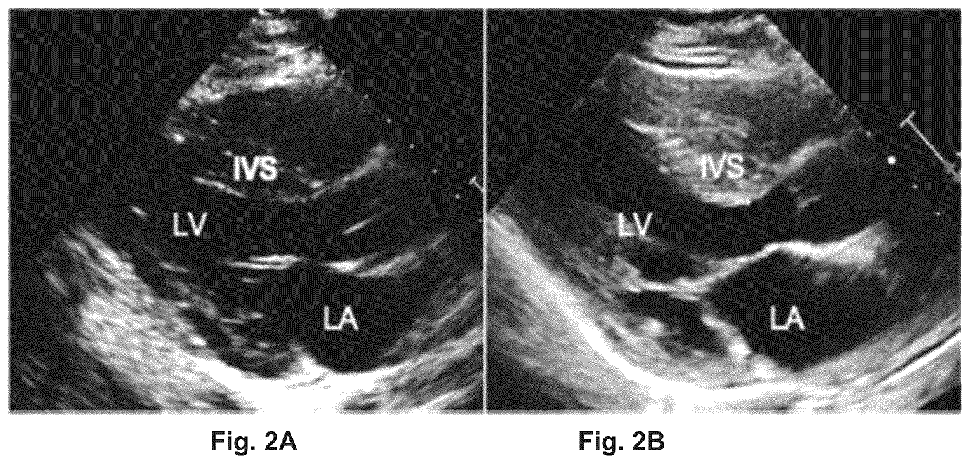

Similarly, HCM is characterized by increased heart wall thickness, smaller cavity size and normal or hyperdynamic left ventricular ejection fraction. FIGS. 2A and 2B show electrocardiogram differences between healthy and unhealthy hearts. Referring to FIGS. 2A and 2B, a transthoracic echocardiogram parasternal long-axis view shows a normal heart (FIG. 2A) vs. a heart with Hypertrophic Cardiomyopathy (FIG. 2B) LA, left atrium; LV, left ventricle; IVS, interventricular septum. In HCM patients, the heart muscle gets thicker and stiffer, making it more difficult to pump blood, which can lead to Sudden Cardiac Death (SCD), heart failure, and/or abnormal heart rhythms causing stroke. Young athletes are particularly at high risk for HCM, and early diagnosis and treatment can prevent fatal complications. Underdiagnosis, delayed diagnosis and barriers to accessing care are contributing factors to adverse outcomes.

As described above, an echocardiogram, an ultrasound image of the heart, is the gold standard for detecting many cardiac conditions including adverse EF and HCM. However, the current procedure for identifying cardiac problems via visual analysis of an echocardiogram takes considerable time, and can be inaccurate with inconsistent diagnoses across different experts. In addition, certain racial backgrounds are more likely to have different patterns of increased heart wall thickness with higher degree cardiac fibrosis/scar burden, which may be challenging to identify. Precise evaluation of cardiac anatomy in the diagnoses of HCM can help tailor treatment approach improves outcomes. Automatic echocardiogram interpretation has the potential to improve HCM detection in community-based settings with limited expertise. Artificial intelligence (AI)-based methods have recently become popular, achieving state-of-the-art in many domains including image and video analyses.

For HCM detection, Deep Video Action Recognition (VAR) models are neural networks architectures that have achieved state-of-the-art performance in recognizing human actions (e.g., running, walking) in a video. VAR models have been successfully applied in a variety of healthcare domains such as care robots, health monitoring, and echocardiogram interpretation] and view classification.

The disclosed approach employs HCM-Dynamic-Echo, an end-to-end deep learning framework to detect HCM, which utilizes a SlowFast Deep VAR model for analyzing a heart echocardiogram. Echocardiogram videos are first segmented into frames, ordered based on their temporal position in the input video, and forwarded to the SlowFast model. SlowFast has two arms: a slow arm captures spatial characteristics, and a second fast arm captures temporal characteristics. Lateral connections integrate characteristics from both arms to enhance spatiotemporal feature learning for video classification. The learned features are then fed into fully connected layers to classify each video as normal or HCM. Additionally, to facilitate training HCM-Dynamic-Echo, in collaboration with cardiac specialists, our team created HypoNet, a large, diverse HCM dataset containing 1, 553 echocardiogram videos. Since HypoNet was not large enough to train HCM-Dynamic-Echo, transfer learning was used. HCM-Dynamic-Echo was pre-trained on 10,030 echo videos in the Stanford EchoNet-Dynamic dataset before fine-tuning it on HypoNet.

Echocardiogram HCM classification is challenging for several reasons. Echocardiographic diagnosis of HCM may overlap with those of other cardiac diseases such as aortic stenosis, hypertension, cardiac amyloidosis, and Fabry disease. Opacification and blurry shapes in images sometimes make accurate analyses challenging. Moreover, it is necessary for experts to make ground truth assessments to guide model development.

Conventional approaches have employed Convolutional Neural Networks (CNNs) to analyze either short video clips or a single view of an echocardiogram in order to derive important cardiac information such as myocardial motion, heart rate and heart rhythm. In contrast, this study uses the SlowFast VAR model to directly evaluate multiple echocardiogram views in 1, 553 echocardiogram videos with the goal of extracting predictive spatiotemporal features and detecting HCM. However, conventional approaches fail to utilize SlowFast on echocardiogram videos for predicting HCM, particularly with a with a sensitivity as disclosed herein.

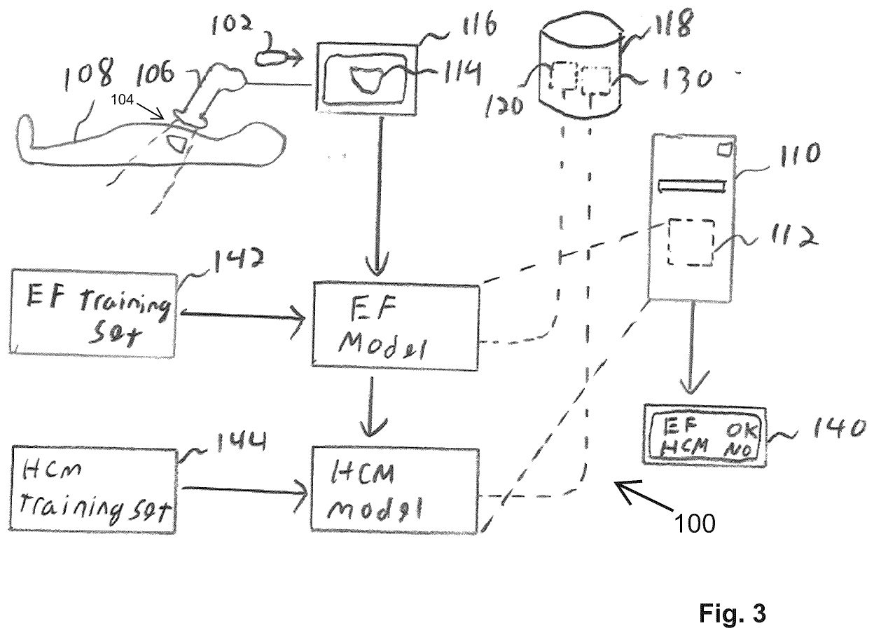

FIG. 3 is a system context diagram including a echocardiogram gathering device and an analysis computer suitable for use with configurations herein. Referring to FIGS. 1-3, a system 100 for analysis of echocardiograms for determining health of a human heart includes receiving a video segment 102 based on an echocardiogram 104, typically administered on a patient 108 using an ultrasound device 106 or similar imaging modality. An analysis server 110 invokes a cardiac application 112 using a stored model 120 for ejection fraction and another model 130 for HCM. The cardiac application 112 trains and stores the models in a storage repository 118, which may be any suitable local storage or cloud service.

The video segment 102 includes renderable images 114 viewable over time on a rendering device 116. The cardiac application 112 compares the video segment 102 to the model of echocardiograms 120, such that the model is trained using labels for a sufficiency of an ejection fraction and to the model 130 for assessing a likelihood of hypertrophic cardiomyopathy (HCM). The cardiac application 112 then renders an indication 140 of a presence of HCM and an indication of an insufficient ejection fraction.

Prior to classification and rendering of the indication, 140, the cardiac application 112 trains the models 120, 130 of echocardiograms, such that training further comprises a corpus of echocardiograms video clips, each clip at least three seconds in duration and capturing at least 5 heartbeats at between 10-50 frames per second. A classification or analysis of a deficient EF includes training, from an ejection fraction training set 142, the ejection fraction model 120. The received video segment 102 is then compared to the ejection fraction model 120, and the cardiac application 112 receives an indication of a deficient ejection fraction depicted by the video segment; and rendering the received indication 140 for use in diagnosis.

Similarly, a classification of HCM, which may include the EF classification, commences with training, from a hypertrophic cardiomyopathy (HCM) training set 144, the hypertrophic cardiomyopathy model 130. The received video segment 102 is compared to the HCM model 130, and the cardiac application 112 receives an indication of a presence of HCM depicted by the video segment 102. The cardiac application 112 then renders the received indication 140 for use in diagnosis.

Comparing the video segment 102 may further include extracting features indicative of spatiotemporal features from the video segment 102, and comparing the extracted features to the model of echocardiograms, including the EF model 120 and the HCM model 130.

In a particular configuration, the cardiac application 112 computes an insufficient ejection fraction based on a binary correspondence with model entries labeled for and insufficient ejection fraction in an EF training set 142. The model 120 may also compute a healthy heart using a dataset split based on ejection fraction.

A corpus of echocardiograms used for training may further include labels indicative of the actual ejection fraction, end systolic volume (ESV), end diastolic volume (EDV) values, frame height and width, frames Per Second (FPS) and number of frames. The corpus of echocardiograms may also define, for each video segment, features indicative of a covariate shift, a presence of black regions, an opacification, and unclear heart linings. Other features include a degree of clearness of heart linings, a contrast, a contraction/relaxation of the heart, and features indicative of a heart wall thickness and a cavity size.

Since the models 120, 130 are based on video segments, rather than individual still images, spatiotemporal activity of the heartbeat may be observed. The ejection fraction model 120 may implement a video action recognition (VAR) neural network. Similarly, the HCM model 130 may implement a slowfast video action recognition (VAR) neural network. The HCM model 130 is configured to perform an analysis using a first slow arm directed at spatial characteristics of the video segment, and a second fast arm directed at temporal characteristics. The HCM model 130 further analyzes spatiotemporal classifiers for a majority averaging for predicting a presence of HCM.

Training the models 120, 130 includes the EchoNet-Dynamic dataset publicly released by Stanford University School of Medicine. EchoNet-Dynamic consists of 10, 030 echocardiograms in Audio Video Interleave (AVI) format with an average length of 3 to 4 seconds. Each video contains 4-5 heartbeats. In addition to the echocardiogram videos, metadata or additional information was avai able including the actual EF, End systolic volume (ESV), End diastolic volume (EDV) values, frame height and width, Frames Per Second (FPS), number of frames and what split the echocardiogram belonged to (train, test or validation set). This metadata was determined by the radiologists using the echocardiograms. The description of these columns is given in Table I.

TABLE I

| Variable | Description |
|---|---|
| FileName | Hashed file name that is used to link clips, |
| | labels, and annotations. |
| EF | Ejection fraction (EF) is determined |
| | using ESV and EDV. |
| ESV | End systolic volume (ESV) is determined |
| | using the discs technique. |
| | End diastolic volume (EDV) is determined |
| EDV | using the discs technique. |
| Height | The height of the video. The |
| Width | width of the video. Frames per |
| | second. |
| FPS | The total number of frames in the video. |
| NumFrames | Type of data split that the video |
| Split | belongs to: Train, Validation,or Test. |

In one approach, training, validation, and test split ratios of 74.4%, 12.8%, and 12.7% respectively were employed, and each split had a prevalence of roughly 22%. Prevalence is the ratio of the number of positive examples to the number of total examples. To create healthy and unhealthy classes, we utilized a threshold of 50 for the EF, as shown in Table II.

TABLE II

| Class number | Formal | number of video | Name |
|---|---|---|---|
| Class 0 | [EF < 50] | 2246 | Unhealthy |
| Class 1 | [EF ≥ 50] | 7784 | Healthy |

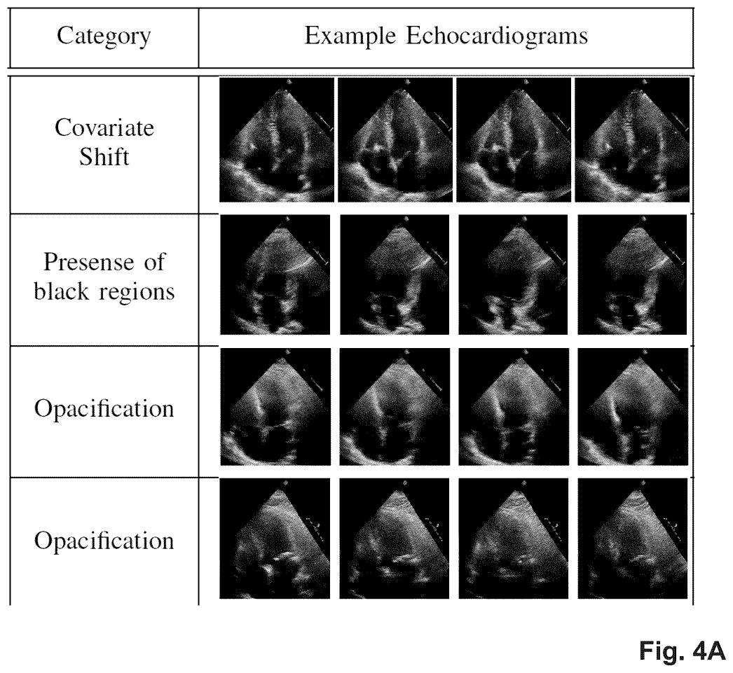

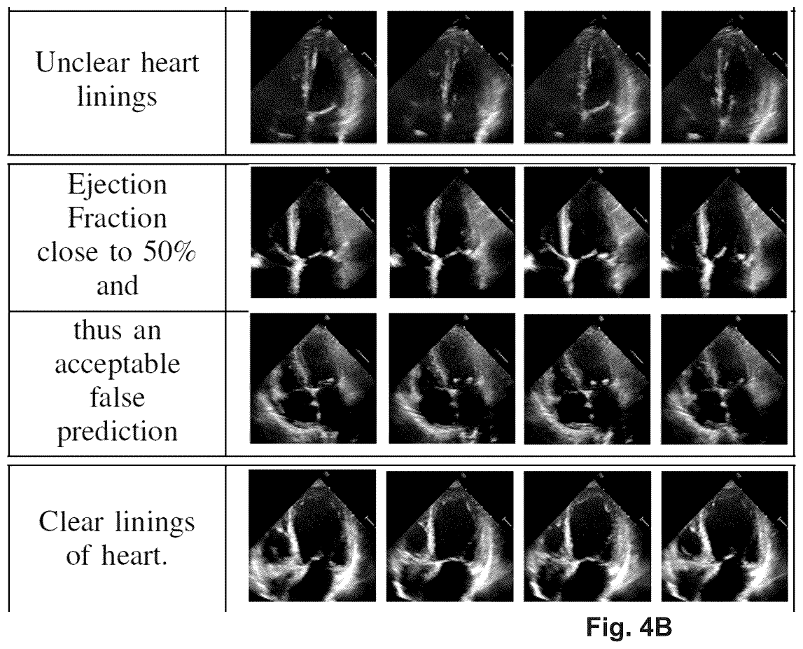

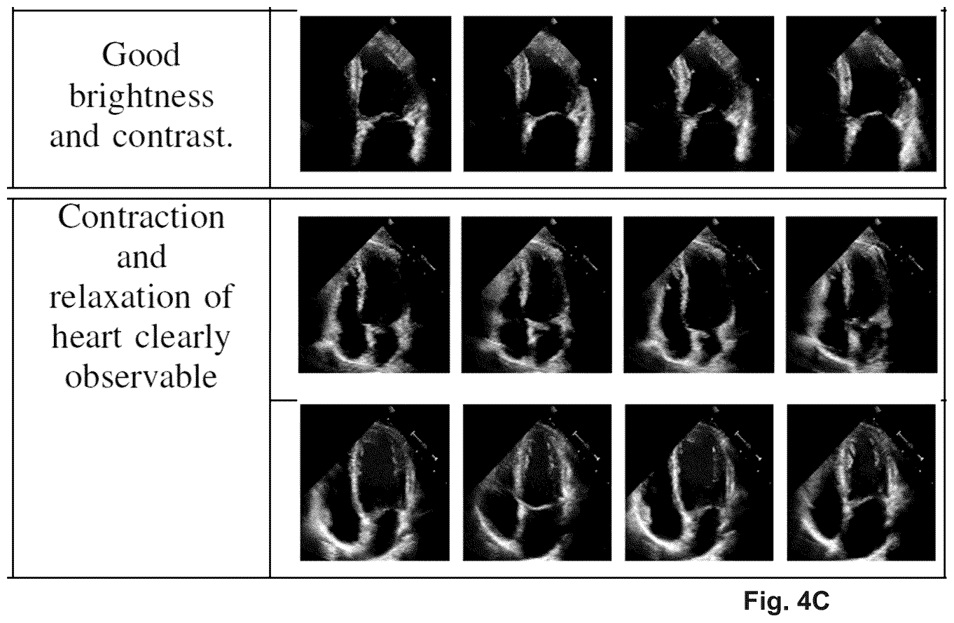

Pixel based scans introduce a number of potential features and feature anomalies. FIGS. 4A-4C show scans exhibiting features employed for classification, and for outlier/border scenarios where artifacts and anomalies can contribute to false positives/negatives. Referring to FIGS. 4A-4C, Imaging technology artifacts: The range of pixel values, and hence the color of the video/frames, were different from that of standard videos. It was speculated that these might be artifacts from medical imaging technologies. This color shift in pixel space may in turn cause a Covariate Shift, a well-known challenge for deep learning classification models, and prevent the accurate classification of the video. Additionally, mechanical issues might have resulted in poor lighting, contrast, and saturation. An example can be seen in the False Positive category in Table IV under the subcategory of Covariate Shift.

Opacification of the heart region: Some of the videos had opacification of the heart region, which contaminated the information in the heart region that is most useful in predicting whether a given heart is healthy or not. Apart from opacification, in some cases, the heart region was covered by black regions, which could also cause the model to make a wrong prediction. These types of videos can be seen in the examples in Table IV under the False Positive category and Opacification, Presence of black regions subcategory.

Unclear heart ventricle linings: The linings of the heart ventricles were not clearly defined in some of the videos, which could also lead to incorrect classification. This artifact could cause the ventricles to appear larger, increasing the EF and predicting it as a positive example of a healthy heart. Examples of videos with unclear heart linings can be seen in Table IV under the False Positive category and unclear heart linings subcategory.

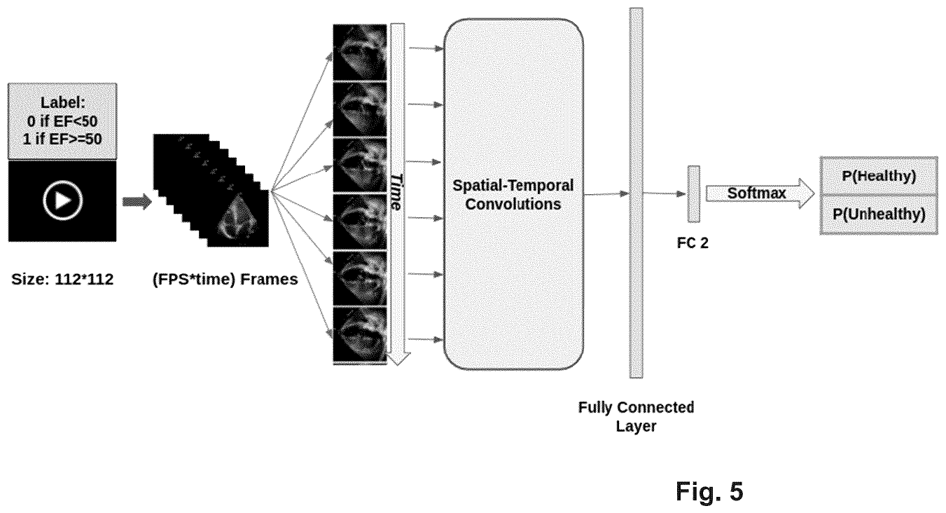

A general workflow of the EF classification pipeline used for the EF model 120 is shown in FIG. 5. A video clip is first separated into numerous frames and then a Spatial-Temporal CNN is used to extract dis-tinct characteristics for cardiac assessment from these frames. These learned features are passed along to the fully connected layer or Softmax layer, which outputs the probabilities that the heart is healthy or unhealthy.

The disclosed EF model 120 invokes a Gate-Shift Module (GSM) for classification based on the ejection fraction. The space-time structure of the video format is one of the most significant challenges in VAR, which requires temporal reasoning for fine-grained recognition. The Gate-Shift Module (GSM), which uses spatial gating in the Spatial-temporal decomposition of 3D kernels, was developed to overcome this issue. GSM begins with a 2D convolution, then separates the output tensor into two tensors using learnable spatial gating: a gated version and its residual. A 1D temporal convolution is applied to the gated tensor, with the residual being skip-connected to the output. Furthermore, GSM's design is based on grouped spatial and spatiotemporal (GST) and Temporal Shift Module (TSM), but instead of a hard-wired channel split, it uses a learnable spatial gating block.

The gate block, when used in conjunction with the fuse block, selectively routes gated features through time shifts and merges them with the spatially convolved residual. To derive the spatial gating planes, GSM employs 2D kernels, time-shifts with no parameter, and a few other parameters.

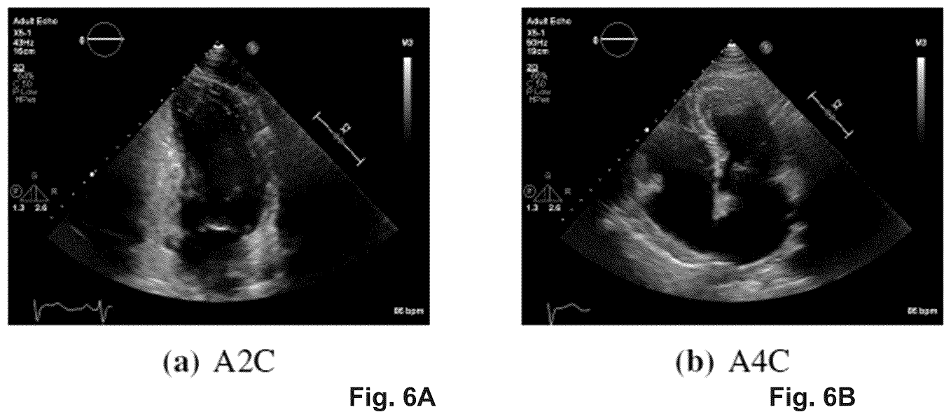

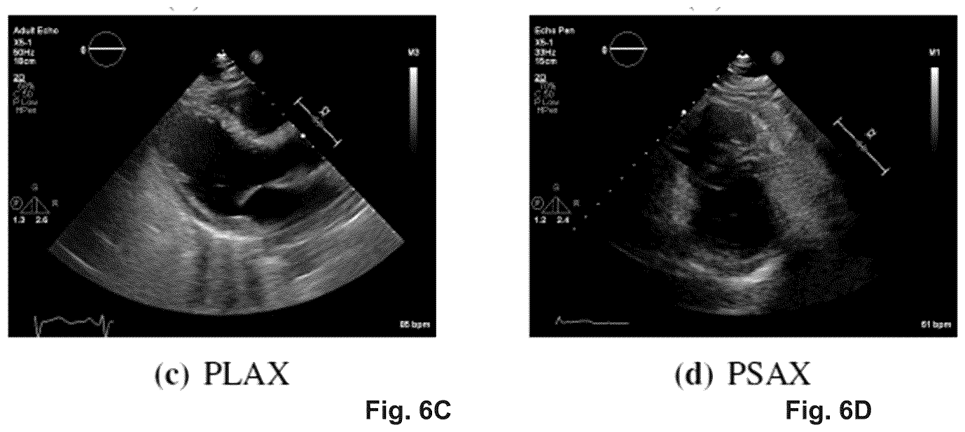

The HCM model 130 invokes an HCM echo dataset, HCM-Net, which was collected through a collaborative effort of cardiac specialists from the HCM Program at Boston Medical Center (BMC) under the Institutional Review Board (IRB) approval number H-44101. The dataset includes 1,553 echocardiogram videos from both HCM patients and controls, recorded between 2016 and 2021. These videos capture diverse cardiac views, including Apical 2-Chamber (A2C), Apical 4-Chamber (A4C), Parasternal Long Axis (PLAX), and Parasternal Short Axis (PSAX), among others, as illustrated in FIGS. 6A-6D. Diagnoses were confirmed by board-certified cardiologists through clinical history, echocardiography, and genetic testing. The dataset was split into training (70%), validation (15%), and testing (15%) sets, maintaining a balanced prevalence of HCM across the partitions.

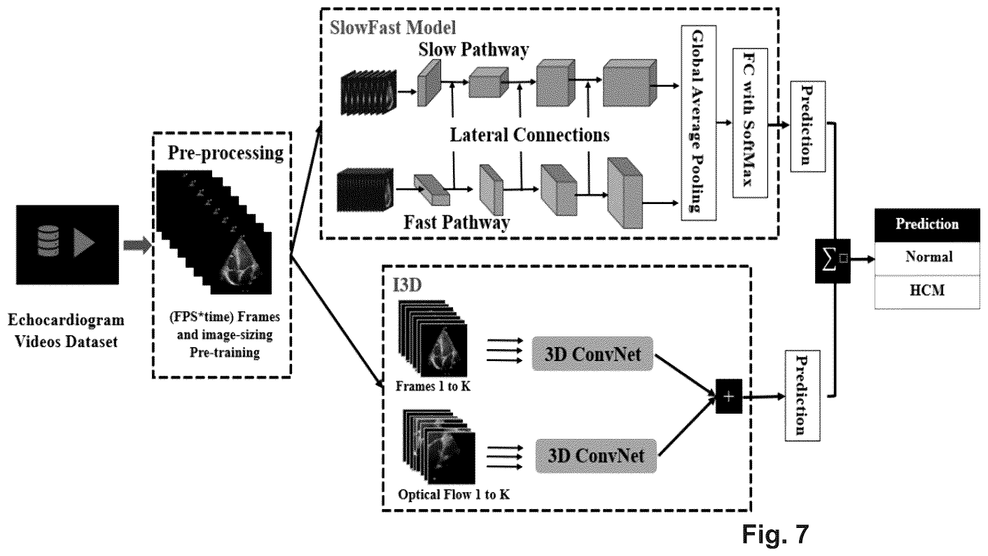

FIG. 7 presents an overview of HCM-Echo-VAR-Ensemble, an ensembled improvement of HCM-Dynamic-Echo, which utilizes only the SlowFast model for HCM detection from echocardiography videos. HCM-Echo-VAR-Ensemble advances this approach by ensembling SlowFast with a second VAR architecture, and employing a majority averaging ensembling method to combine both models, thus improving HCM classification performance. HCM-Echo-VAR-Ensemble innovatively combines the best of both models, leveraging the single-pathway architecture of I3D and the dual-pathway architecture of SlowFast to achieve more accurate classification. Referring to FIG. 7, the HCM-Echo-VAR-Ensemble framework employs a deep VAR models for HCM classification. As a pre-processing step, all VAR models were pre-trained on a large EchoNet Dynamic dataset to address the data scarcity issue. Thereafter, all models were fine-tuned on our smaller HCM dataset to facilitate accurate classification of test echocardiograms as HCM vs. no HCM. I3D provides efficient spatial modeling and parallel processing, while SlowFast captures both slow and fast motion patterns with its asymmetric design. By integrating their predictions through majority averaging as in the equation below, the ensemble overcomes the limitations of I3D in modeling complex long-term temporal dependencies.

$$EP ( x ) = arg {max}_{c} ( {\sum }_{i = 1}^{N} I ( {M}_{i} ( x ) = c ) )$$

where argmax_{c} returns the class label with the highest sum of predictions, and I(Mᵢ(x)=c) is an indicator function that returns 1 if Mᵢ(x) predicts class c for input x, and 0 otherwise.

This method leverages the strengths of both models for superior accuracy in HCM detection. HCM-Echo-VAR-Ensemble is rigorously evaluated to demonstrate that the ensemble outperforms both of the individual I3D and SlowFast VAR models for the task of HCM diagnosis in echocardiograms, with an accuracy of 95.28% and a sensitivity of 93.97%, compared to 86.70% accuracy and 87.07% sensitivity for I3D, and 93.13% accuracy and 91.38% sensitivity for SlowFast.

SlowFast uses ResNet 3D as a backbone architecture, which employs 3D convolutional layers optimized for efficient video recognition. SlowFast, shown in FIG. 7, has two main pathways. The “slow” pathway has a large stride and analyzes each frame at high resolution to capture spatial semantics despite its low frame rate and temporal resolution. The “fast” pathway has a smaller stride and processes frames at a high rate and a higher temporal resolution to capture motion at fine temporal resolutions. Both pathways are combined by lateral connections. The features from each path are pooled using a global average pooling layer and combined before being fed to the fully connected layer. Finally, the softmax function:

$$\frac{exp ( {x}_{i} )}{{\sum }_{j} exp ( {x}_{j} )}$$

is used for classification. Experiments on state-of-the-art video recognition benchmarks shows that SlowFast performs well for video recognition.

The Echonet dataset consists of 10,030 echo video clips, each labeled as having a healthy or abnormal ejection fraction (EF). First, multiple frames are extracted from EchoNet video clips and ordered based on their temporal position in the video. These frames are then input to the SlowFast VAR model, which captures spatiotemporal characteristics of the echo video. The learned features are then fed to fully connected layers, followed by a softmax to classify the heart as either having a healthy EF (probability≥50%) or unhealthy (probability<50%).

After pre-training, transfer learning is used to train the SlowFast model, addressing challenges such as limited data and sparse occurrence of multiple echocardiogram views in HypoNet. After the SlowFast model is fine-tuned on HypoNet, the learned features are passed through fully connected layers. Finally, the output of the fully connected layer is passed through a softmax to classify whether the input video clip represents a normal heart or one with HCM.

Those skilled in the art should readily appreciate that the programs and methods defined herein are deliverable to a user processing and rendering device in many forms, including but not limited to a) information permanently stored on non-writeable storage media such as ROM devices, b) information alterably stored on writeable non-transitory storage media such as solid state drives (SSDs) and media, flash drives, floppy disks, magnetic tapes, CDs, RAM devices, and other magnetic and optical media, or c) information conveyed to a computer through communication media, as in an electronic network such as the Internet or telephone modem lines. The operations and methods may be implemented in a software executable object or as a set of encoded instructions for execution by a processor responsive to the instructions, including virtual machines and hypervisor controlled execution environments. Alternatively, the operations and methods disclosed herein may be embodied in whole or in part using hardware components, such as Application Specific Integrated Circuits (ASICs), Field Programmable Gate Arrays (FPGAs), state machines, controllers or other hardware components or devices, or a combination of hardware, software, and firmware components.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A method for analysis of echocardiograms for determining health of a human heart, comprising:
receiving a video segment based on an echocardiogram;
comparing the video segment to a model of echocardiograms, the model trained using labels for a sufficiency of an ejection fraction and a likelihood of hypertrophic cardiomyopathy (HCM); and
rendering an indication of a presence of HCM and an indication of an insufficient ejection fraction.

2. The method of claim 1 wherein the insufficient ejection fraction is computed based on a binary correspondence with model entries labeled for insufficient ejection fraction.

3. The method of claim 1 computing a healthy heart using a dataset split based on the ejection fraction.

4. The method of claim 1 wherein comparing further comprises:
extracting features indicative of spatiotemporal features from the video segment; and
comparing the extracted features to the model of echocardiograms.

5. The method of claim 1 further comprising training the model of echocardiograms, training further comprising a corpus of echocardiograms video clips, each clip at least three seconds in duration and capturing at least 5 heartbeats at between 10-50 frames per second.

6. The method of claim 5 wherein the corpus of echocardiograms further include labels indicative of the actual ejection fraction, end systolic volume (ESV), end diastolic volume (EDV) values, frame height and width, frames Per Second (FPS) and number of frames.

7. The method of claim 5 wherein the corpus of echocardiograms define, for each video segment, features indicative of a covariate shift, a presence of black regions, an opacification, and unclear heart linings.

8. The method of claim 5 wherein the corpus of echocardiograms define, for each video segment, a degree of clearness of heart linings, a contrast, and a contraction/relaxation of the heart.

9. The method of claim 5 wherein the corpus of echocardiograms further include features indicative of a heart wall thickness and a cavity size.

10. The method of claim 5 further comprising:
training, from an ejection fraction training set, an ejection fraction model;
comparing the video segment to the ejection fraction model;
receiving an indication of a deficient ejection fraction depicted by the video segment; and
rendering the received indication for use in diagnosis.

11. The method of claim 5 further comprising:
training, from a hypertrophic cardiomyopathy (HCM) training set, a hypertrophic cardiomyopathy model;
comparing the video segment to the HCM model;
receiving an indication of a presence of HCM depicted by the video segment; and
rendering the received indication for use in diagnosis.

12. The method of claim 10 wherein the ejection fraction model implements a video action recognition (VAR) neural network.

13. The method of claim 11 wherein the HCM model implements a slowfast video action recognition (VAR) neural network.

14. The method of claim 13 wherein the HCM model performs analysis using a first slow arm directed at spatial characteristics of the video segment, and a second fast arm directed at temporal characteristics.

15. The method of claim 14 wherein the HCM model further analyzes spatiotemporal classifiers for a majority averaging for predicting a presence of HCM.

16. A computer program embodying program code on a non-transitory computer readable storage medium that, when executed by a processor, performs steps for implementing a method for analysis of echocardiograms for determining health of a human heart, the method comprising:
receiving a video segment based on an echocardiogram;
comparing the video segment to a model of echocardiograms, the model trained using labels for a sufficiency of an ejection fraction and a likelihood of hypertrophic cardiomyopathy (HCM); and
rendering an indication of a presence of HCM and an indication of an insufficient ejection fraction.
